(19) United States
(12) Patent Application Publication
(10) Pub. No.: US 2013/0204351 A1
(43) Pub. Date: Aug. 8, 2013

(54) Aneurysm Graft Devices And Methods

(71) Applicant: Inceptus Medical LLC, Aliso Viejo, CA (US)

(72) Inventors: Brian J. Cox, Laguna Niguel, CA (US); Robert F. Rosenbluth, Laguna Niguel, CA (US); Paul Lubock, Monarch Beach, CA (US)

(73) Assignee: Inceptus Medical LLC, Aliso Viejo, CA (US)

(21) Appl. No.: 13/758,957

(22) Filed: Feb. 4, 2013

Related U.S. Application Data
(60) Provisional application No. 61/594,135, filed on Feb. 2, 2012.

(51) Int. Cl. A61F 2/852

(52) U.S. Cl. CPC A61F 2/852
USPC 623/1.21; 623/1.35; 623/1.3

(57) ABSTRACT

The present invention provides methods and apparatus for the endoluminal positioning of an intraluminal prosthesis at a target location within a body lumen. The device may comprise a porous, multi-layer prosthesis that can include several different tubular mesh components such as an everted tubular component, an tubular component that expands to various different sizes, a bifurcated tubular component, and a tubular component with an undulating expanded shape, among others. Various components can have different densities or pore sizes and can be overlaid on each other to selectively block different regions of a vessel.

### RELATED APPLICATIONS

This application claims priority to U.S. Provisional Application Ser. No. 61/594,135 filed Feb. 2, 2012 entitled Aneurysm Graft Devices and Methods, which is hereby incorporated herein by reference in its entirety.

### BACKGROUND OF THE INVENTION

The present invention relates generally to methods and apparatus for the endoluminal placement of tubular prostheses, such as grafts, stents, and other structures. More particularly, the present invention relates to the implantation of luminal prostheses within a body lumen to treat a vascular defect, such as an aortic aneurysm, an aortic dissection, a thoracic aneurysm, and a thoracic dissection.

Vascular aneurysms 10 are the result of abnormal dilation of a blood vessel, usually resulting from disease and/or genetic predisposition which can weaken the arterial wall and allow it to expand. While aneurysms can occur in any blood vessel, most occur in the brain, aorta and peripheral arteries, with the majority of aortic aneurysms occurring in the abdominal aorta 12, usually beginning below the renal arteries 14 and often extending distally into one or both of the iliac arteries 16 as shown in FIG. 1. The thoracic aorta (shown in FIG. 24), is also a common location of aneurysm occurrence, usually involving a weakening of the aortic wall associated with connective tissue disorders like the Marfan and Ehler-Danlos syndromes or congenital bicuspid aortic valve.

In the past, most aortic aneurysms were treated in open surgical procedures where the diseased vessel segment is bypassed and repaired with an artificial vascular graft. While considered to be an effective surgical technique, particularly considering the alternative of a usually fatal ruptured abdominal aortic aneurysm, conventional vascular graft surgery suffers from a number of disadvantages. The surgical procedure is complex and requires experienced surgeons and well-equipped surgical facilities. Even with the best surgeons and equipment, however, the patients being treated frequently are elderly and weakened from cardiovascular and other diseases, reducing the number of eligible patients. Even for eligible patients, conventional aneurysm repair surgery performed prior to rupture has a relatively high mortality rate, usually from 2% to 10%. Morbidity related to the conventional surgery includes myocardial infarction, renal failure, impotence, paralysis, and other conditions. Additionally, even with successful surgery, recovery can take several weeks and often requires a lengthy hospital stay.

Aortic dissection, such as abdominal aortic dissection or thoracic aortic dissection, occurs when a tear 50 in the inner wall of the aorta 52 causes blood to flow between the layers of the wall 54 of the aorta, forcing the layers apart (e.g., see the thoracic aortic dissection in FIG. 24). The dissection typically extends anterograde, but can extend retrograde from the site of the intimal tear. Aortic dissection is a medical emergency and can quickly lead to death, even with optimal treatment. If the dissection tears the aorta completely open (through all three layers), massive and rapid blood loss occurs. Aortic dissections resulting in rupture have an 80% mortality rate, and 50% of patients die before they even reach the hospital. All acute ascending aortic dissections require emergency surgery to prevent rupture and death.

In order to overcome some or all of these drawbacks, endovascular stent-graft placement procedures for the treatment of aneurysms or dissections have become increasingly common. Generally, such endovascular procedures will deliver a radially compressed stent-graft intravascularly and extending through the vascular defect. The graft is then expanded in situ, either by releasing a self-expanding graft or by internally expanding a malleable graft (e.g. using a balloon catheter) to exclude the dilated aneurysmal portion of the artery from flow and pressure. Typically, commercially available stent-grafts comprise both a frame and a liner, where the frame provides the necessary mechanical support and the liner provides the necessary blood barrier.

Present endovascular stent-grafts for vascular defect repair, however, suffer from a number of limitations. A significant number of endoluminal repair patients experience leakage at the proximal juncture (attachment point closest to the heart) within two years of the initial repair procedure. While such leaks can often be fixed by further endoluminal procedures, the need to have such follow-up treatments significantly increases cost and is certainly undesirable for the patient. A less common but more serious problem has been graft migration. In instances where the graft migrates or slips from its intended position, open surgical repair is required. This is a particular problem since the patients receiving the endoluminal stent-grafts are those who are not considered good candidates for open surgery. Further shortcomings of the present endoluminal stent-graft systems relate to achieving a stable positioning within aneurysms having torturous geometries.

The following references relate to the present invention and are hereby incorporated by reference herein: U.S. Pat. Nos. 4,954,126; 5,741,325; 5,951,599; 6,942,693; 7,588,597; 8,182,506; 8,261,648; 8,267,986; U.S. Pub. Nos. 2010/00305686; 2003/0014075; and Geremia et al. (2000, April). Occlusion of Experimentally Created Fusiform Aneurysms with Porous Metallic Stents. AJNR AM J Neuroradiol 21:739-745, April 2000; Henry et al. (2008). Treatment of Renal Artery Aneurysm With the Multilayer Stent. J Endovasc Ther 15:231-236; and Polydorou et al. (2012). Endovascular Treatment of Aortic Aneurysms: the Role of the Multilayer Stent. Hospital Chronicles 2012, Vol. 7, Supp. 1:157-159.

### SUMMARY OF THE INVENTION

The present invention provides methods and apparatus for the endoluminal positioning of an intraluminal prosthesis at a target location within a body lumen. The prosthesis is suitable for a wide variety of therapeutic uses, including stenting of the ureter, urethra, biliary tract, and the like. The devices and methods will also find use in the creation of temporary or long-term lumens, such as the formation of fistulas. In particular, the device is useful for implantation into blood vessels for the treatment of vascular defects, such as aneurysms (e.g., aortic or thoracic), dissections (e.g., aortic or thoracic), vascular stenoses, and the like. In some embodiments, the device may comprise a porous, multi-layer prosthesis. In some embodiments, the layers of the prosthesis may serve to segment the vascular treatment site thus forcing flow to cross a plurality of layers to reach the vascular wall such that it embolizes progressively from the outside in toward the inner most layer which defines a new vascular lumen. Thus, the multi-layer prosthesis may serve to rapidly protect the outmost areas of an aneurysm or other vascular defect first. As the embolization progresses and occludes the inner most layer(s), the construction provides a matrix for healing of the prosthesis.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 illustrates an example aorta having a vascular aneurysm;

FIGS. 2-3 illustrate an embodiment of an everted mesh prosthesis according to the present invention;

FIG. 4 illustrates another embodiment of a prosthesis having multiple eversions according to the present invention;

FIG. 5 illustrates an embodiment of a tubular mesh prosthesis component according to the present invention;

FIG. 6 illustrates an embodiment of a flared mesh prosthesis component according to the present invention;

FIG. 7 illustrates an embodiment of a D-shaped mesh prosthesis component according to the present invention;

FIG. 8 illustrates the prosthesis components of FIGS. 4, 6, and 7 according to the present invention;

FIGS. 9A and 9B illustrate an embodiment of a bifurcated mesh component according to the present invention;

FIGS. 10A-10D illustrate an embodiment and deployment of a prosthesis with four layer components according to the present invention;

FIG. 11 illustrates an embodiment of a mesh prosthesis component having an undulating expanded shape according to the present invention;

FIG. 12 illustrates an example aorta having a vascular aneurysm;

FIG. 13 illustrates an embodiment of a mesh prosthesis component having an expanded shape sized to contact the walls of an aneurysm according to the present invention;

FIG. 14 illustrates an embodiment of a mesh according to the present invention;

FIG. 15 illustrates an embodiment of a mesh prosthesis component being woven on a mandrel according to the present invention;

FIG. 16 illustrates an embodiment of mesh according to the present invention;

FIG. 17 illustrates an embodiment of a mesh component having engagement members for attaching to a second mesh prosthesis component according to the present invention;

FIGS. 18A, 18B, 19A, and 19B illustrate embodiments of an engagement member according to the present invention;

FIG. 20 illustrates an embodiment of an engagement member ring according to the present invention;

FIG. 21 illustrates an embodiment of mesh having integrated engagement members according to the present invention;

FIG. 22 illustrates an embodiment of an engagement member according to the present invention;

FIG. 23 illustrates an embodiment of a prosthesis having a low density prosthesis layer and a high density prosthesis layer according to the present invention;

FIG. 24 illustrates an embodiment of a single layer prosthesis component having both low density portions and high density portions according to the present invention;

FIG. 25 illustrates an embodiment of a first mesh prosthesis layer folded around the end of a second mesh prosthesis layer according to the present invention; and,

FIGS. 26A-26C illustrate a method of filling an aneurysm according to the present invention.

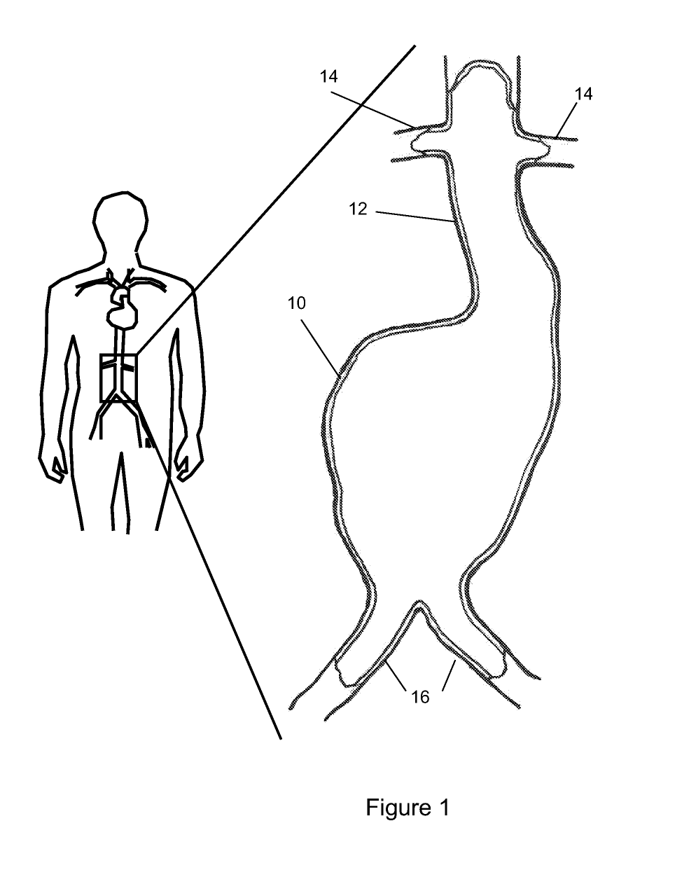

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

While the present invention is illustrated in connection with treatment of abdominal aortic aneurysms and thoracic aortic aneurysms, it should be understood that abdominal aortic dissections, thoracic aortic dissections, and similar vascular defects can also be treated with the embodiments of the present invention.

Generally, the present invention includes embodiments with one or more high density, low porosity mesh prosthesis components. As described in more detail elsewhere in this specification, the density is such that it forms an effective sealing barrier within the patient for correcting various vascular defects. While prior art devices have focused on the use of both a rigid stent structure for support combined with a low porosity fabric or polymer layer for sealing purposes, the high density and/or low pore-size mesh can provide enough structural rigidity to be deployed individually while also providing the barrier or sealing functionality often provided by fabric-type layers. In this respect, the high density components of the present invention can be deployed alone or in connection with less dense layers (i.e., onto or under other component layers). Additionally, this single layer can be relatively thin and reduced to a relatively small diameter, allowing it's deployment from catheters that are generally smaller than those used with other prior art vascular treatment devices. Furthermore, the mesh braiding allows a single layer to be varied in density/pore size along a component's length, allowing some regions to exhibit varying levels of occlusion or even a total lack of occlusion.

In some embodiments, it may be desirable to load multiple components or layers onto a single deployment catheter and deliver these components within a patient at the same time. These components may be simply placed over each other in a desired orientation or can be further attached to each other, as discussed in detail elsewhere in this specification. In other embodiments, it may be desirable to deploy multiple components successively, such as via different delivery catheters.

The multi-layered prosthesis embodiments of the present invention may be constructed as one multi-layered structure or built in-vivo using a plurality of component pieces. One potential advantage of in-vivo construction is that it allows for the use of a smaller delivery catheter than would be necessary if a multi-layer device were constructed outside the body and then inserted in as one piece.

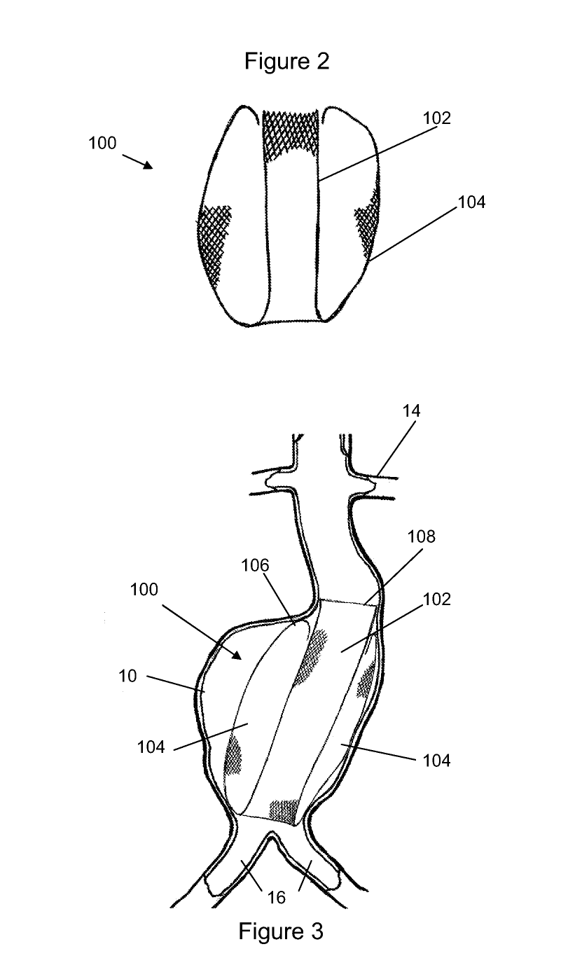

One portion or component of the multi-layer construction may be achieved by everting or folding a porous tubular mesh to form a multi-layer structure, either within a patient or prior to insertion into a patient. For example, the embodiment of FIG. 2 illustrates an everted tubular prosthesis 100 having an inner tubular region 102 and an outer everted portion 104 forming a torus or donut-like shape. As seen in FIG. 3, the outer everted portion 104 can provide a lining of a substantial portion of the aneurysm 10, while the inner tubular region 102 forms an inner lumen or passage. Thus, the everted prosthesis 100 provides a luminal construct that can either stand alone or can support additional components or layers, as discussed further below.

In this example, the proximal end 106 of the prosthesis 100 is positioned near the top of the aneurysm 10, however, the end 106 may also be positioned at any position along the length of the aneurysm 10 (e.g., three-fourths, halfway, or a quarter of the way down the aneurysm 10). Similarly, the distal end 108 may be located at the top of the aneurysm 108 as shown in FIG. 5 or can extend to any number of positions further up the aorta, such just below the renal arteries 14 or past/covering the renal arteries 14.

In some embodiments, an everted prosthesis or component may be macroporous with an average mesh pore size that is larger than about 300 microns. In some embodiments, an everted component may have an average pore size of between about 300 and 1500 microns.

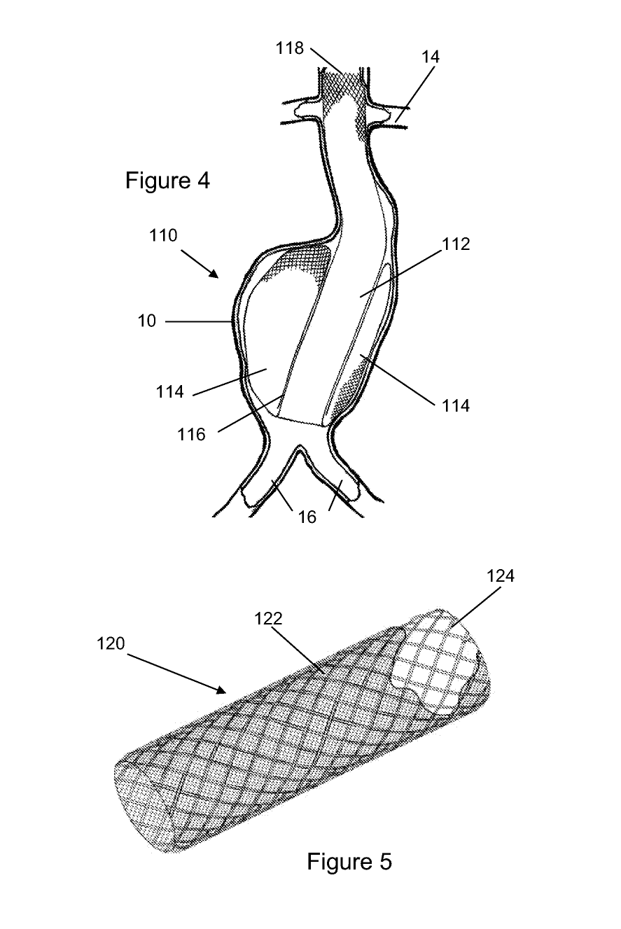

The prosthesis may be everted or folded once, as seen with the prosthesis 100 of FIGS. 2 and 3, or may be everted/folded several times to create a plurality of layers within the patient. For example, FIG. 4 illustrates a prosthesis 110 that is generally similar to the previously described prosthesis 100, but has an increased length to allow for a secondary eversion or fold. Specifically, the everted portion 114 is formed by the proximal end 116 being positioned near a bottom of the aneurysm 10, extending to the top of the aneurysm 10, and then looping back down to the bottom of the aneurysm 10 again. In other words, the everted portion 114 is folded once at each end of the aneurysm 10 such that the proximal end 116 is located near the bottom of the prosthesis 110 and three discrete layers are formed within the aneurysm 10.

As also seen in FIG. 4, the inner tubular region 112 is elongated such that its distal end 118 extends past the renal arteries 14 (transrenal or suprarenal). Preferably, the mesh of the distal end 118 of the inner tubular region 112 is porous enough to allow blood flow through the renal arteries 114. In one example, the average mesh pore size of a suprarenal component or portion may be greater than about 150 microns. In another example, a suprarenal component or portion may have an average mesh pore size of between about 200 and 800 microns. In another example, a suprarenal portion or component may have an average pore size of between about 300 and 1500 microns. The pore size, also referred to as the effective pore size, is defined as the largest circle 216 that can be inscribed within an opening of the fibers 212, 214 of the mesh structure 210 as shown in FIG. 16. Alternately, the mesh may include apertures or gaps positioned to align with the arteries 14, as also discussed in other embodiments in this specification.

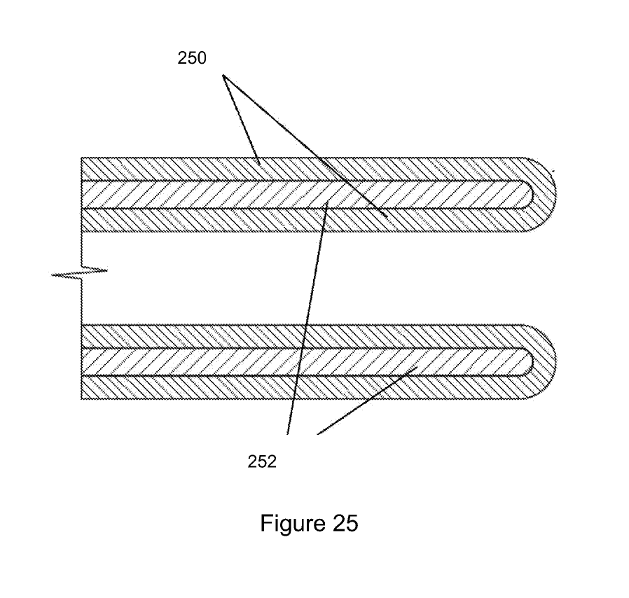

FIG. 25 illustrates another example of an everted or folded prosthesis layer 250. Specifically, the prosthesis layer 250 forms a first or bottom layer and then folds around an end (distal or proximal) of a second prosthesis layer 252. The layer 250 can entirely cover the layer 252, partially cover layer 252, or any combinations of both on the inner/outer surfaces. Hence, the first prosthesis layer 250 at least partially forms both an inner surface and an outer surface of the prosthesis.

Preferably, the previously described everted components 100, 110, and 250 are heat set prior to delivery to form a fold or eversion at a desired location when in an expanded or unconstrained position. If the component is to be folded or everted prior to being loaded onto a delivery catheter, the heat set folds help maintain the everted shape of the component after delivery within a patient. If the component is to be folded within a patient during delivery, the heat set folds help spontaneously form the desired eversion(s) as the component is expanded. It should be further noted that while relatively simple everted shapes, such as that of component 100, can be everted within a patient, more complex everted shapes, such as that of component 110 having multiple folds, are more reliably folded prior to being loaded onto a delivery catheter.

As previously discussed, additional, discrete components can be attached to the previously described mesh prosthesis embodiments 100, 110. For example, FIG. 5 illustrates a generally tubular-shaped mesh component 120 that can be attached within the prosthesis 100 or 110. The component 120 is preferably composed of a mesh formed from either filaments of substantially the same diameter or transverse dimension, or the mesh may be constructed using filaments of different diameter size (e.g., larger diameter filaments 124 and smaller diameter filaments 122).

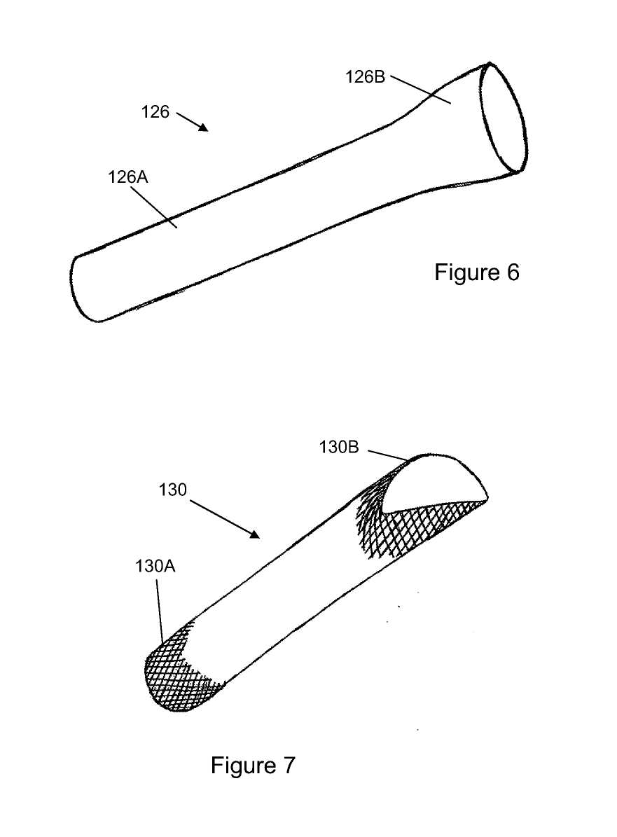

In another example, FIG. 6 illustrates another mesh component 126 having a generally tubular body portion 126A and an outwardly-flared end portion 1268. FIG. 7 illustrates yet another mesh component 130 having a generally tubular body portion with a one end 130A being circular (e.g., tapered, flared, or of contestant size) and a half-circle or “D” shaped end 1308.

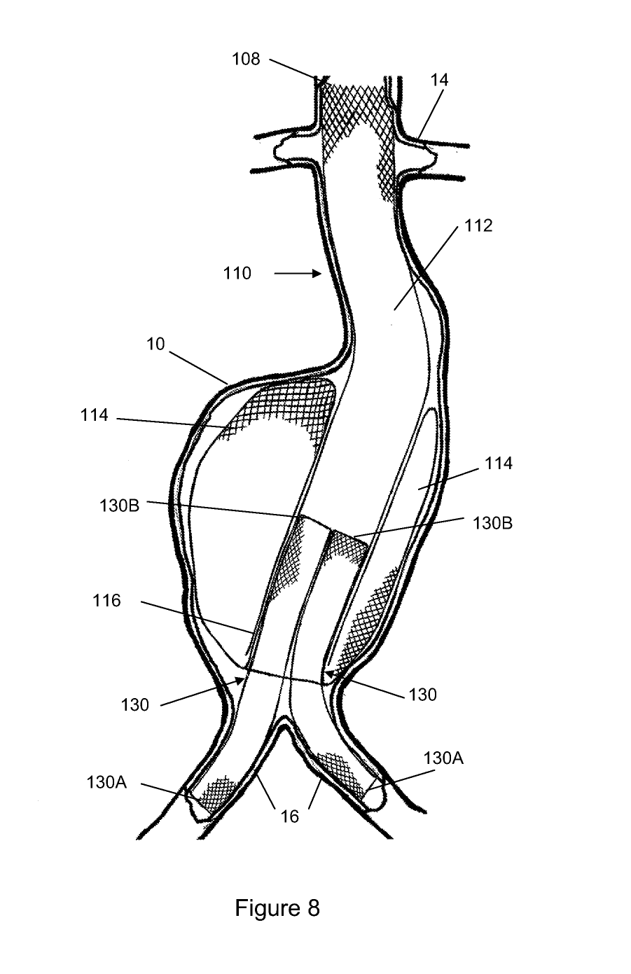

The previously described components 120, 126, and 130 can be used, for example, to treat abdominal aneurysms that have involvement with narrower arteries, such as the iliac arteries 116. For example, in FIG. 8 two D-shaped components 130 are located within the inner tubular region 112 of prosthesis 110 and further extend out of the prosthesis 110 and into the iliac arteries 116. In this respect, the flat regions of their D-shaped ends 130B can be fixed against each other to form an overall circular diameter within the tubular region 112. The distal end of the D-shaped components may have features to facilitate the connection of the two components to each other and the prosthesis 110 (e.g., hooks, barbs, Velcro, such as those described later in this specification).

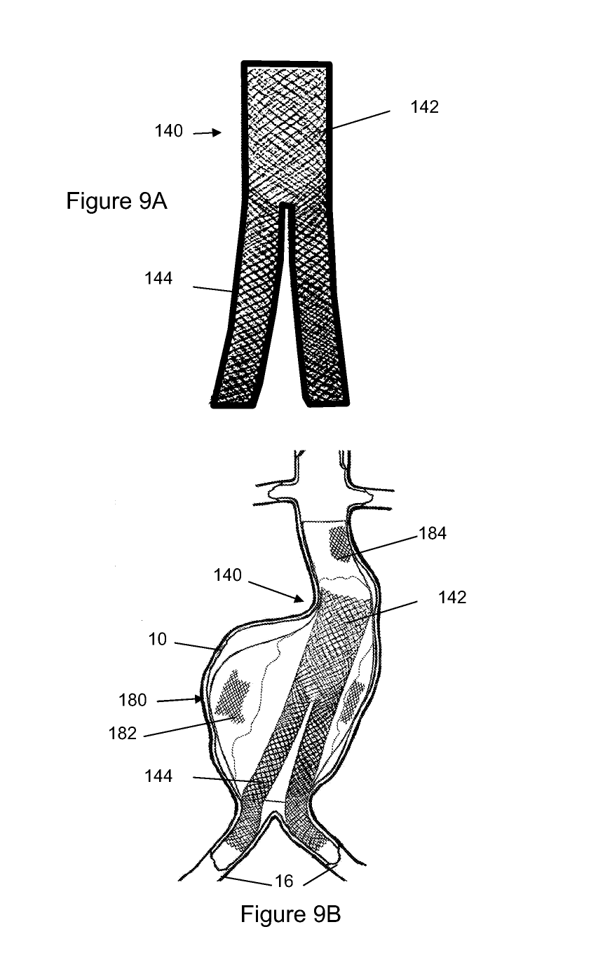

FIGS. 9A and 9B illustrate a bifurcated mesh component 140 having a larger tubular portion 142 that branches into two smaller portions 144. Preferably, the bifurcated component 140 is attached to or within another prosthesis component, such as components 100, 110, or 140, by one of the various attachment mechanisms discussed in this specification. In some embodiments, the bifurcated component 140 is composed of mesh formed from a braid of filaments. A braided bifurcated device and methods of its manufacture are disclosed by Chouinard et al. in U.S. Pat. No. 6,942,693 which is herein incorporated in its entirety by reference.

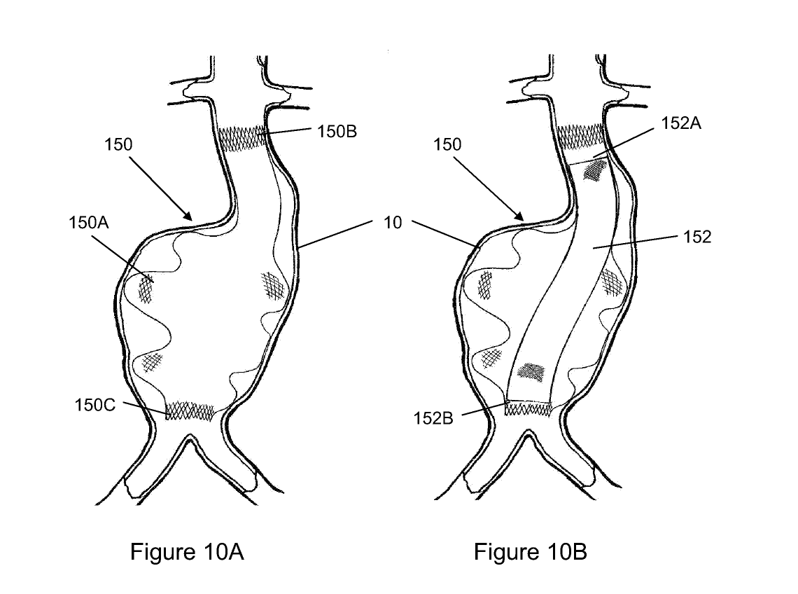

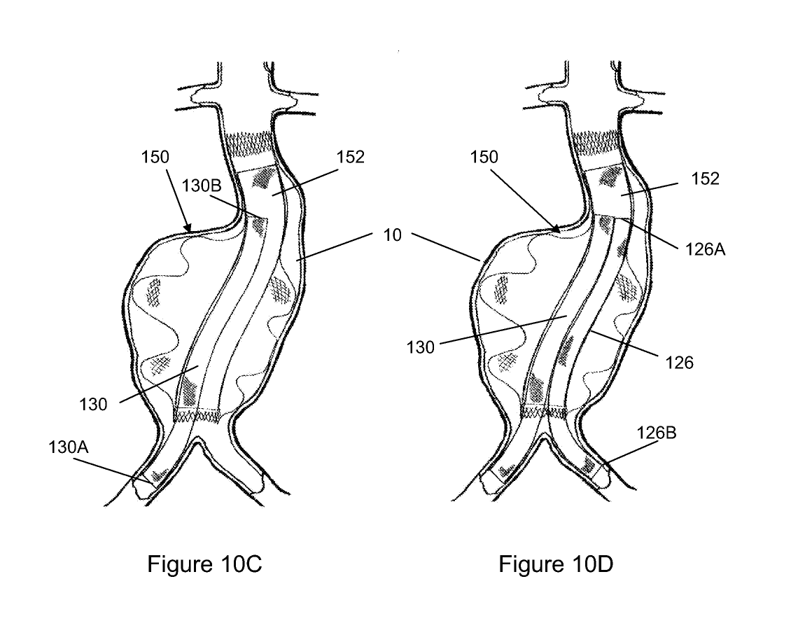

In some embodiments, the porous, multi-layer prosthesis construction may be achieved by the co-axial arrangement of several independent mesh tubes. The mesh tubes may be attached to each other and implanted as one multi-layer device or implanted individually in sequence such that there is a frictional or mechanical engagement of the layers. One example sequential arrangement of prosthesis components are shown in FIGS. 10A through 10D.

In FIG. 10A, a first prosthesis component 150 is deployed within the aneurysm 10. This first prosthesis component 150 includes a distal end 150B and a proximal end 150C which both have a generally narrow diameter when expanded. The middle region 150A has a larger expanded diameter than ends 150A and 150C and may form an undulating or wave-like pattern to reconstruct at least part of the lumen within the aneurysm 10. However, it should be understood that any of the other outer prosthesis components described in this specification can also be used, such as 100, 110, or 140.

FIG. 10B illustrates a tubular prosthesis component 152, which has a generally constant expanded diameter. The distal end 152A and proximal end 152B are located near the distal and proximal ends 150B and 150C or the component 150, respectively, and thereby form the central, reconstructed lumen of the artery.

In FIG. 10C, a first leg component 130 is deployed within the component 152. A D-shaped distal end 130B of the leg component 130 may be deployed inside or upstream of the aneurysm 10 and a proximal end 130A may extend into a downstream branch artery such as an iliac artery. Turning to FIG. 10D, a second leg component 126 is deployed within the component 152 in a similar, but opposing position to component 130, such that a flaring distal end is 126A is located upstream of the aneurysm 10 and the proximal end 130 is located in downstream artery branch.

The leg components in this example include components 130 and 126, it should be understood that any combination of leg components discussed in this specification can be deployed. For example, two components 130 or 126 can be used. Preferably, the first prosthesis component 150 is constructed so as to provide a substantial amount of anchoring force, such as with relatively large diameter filaments and/or with a weaving pattern that creates relatively large diameter pore size. Conversely, the components 152, 130 and 126 preferably have a small average and maximum pore size that can be formed from relatively smaller filaments. In this respect, the first prosthesis 150 acts as an anchoring layer that provides the mechanical support to prevent migration and kinking of the prosthesis as a whole, while components 152, 130 and 126 reconstruct the inner aorta lumen by creating rapid hemostasis leading to occlusion of the aneurysm 10.

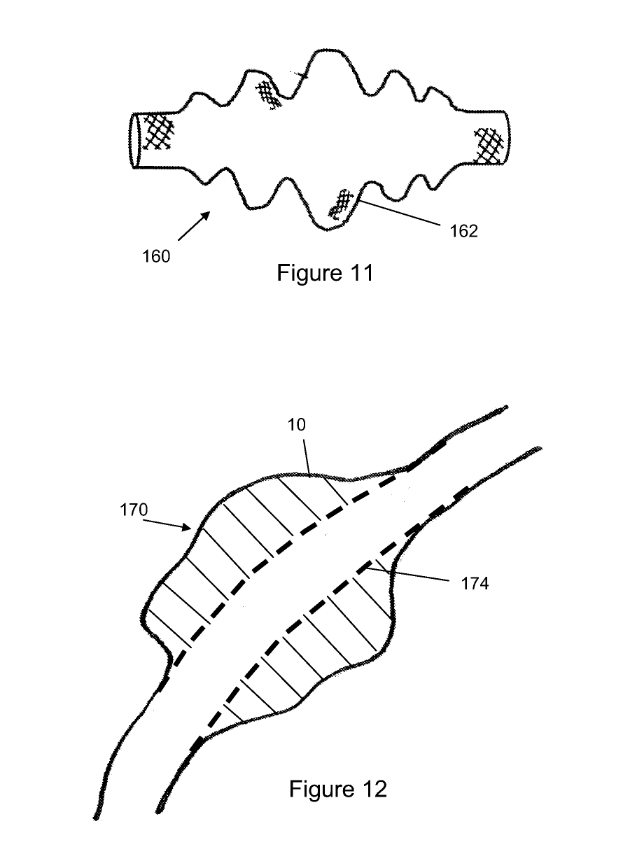

In some embodiments, one or more of the layers or components may be heat set to form radial undulations, diameter changes, wrinkles, dilations or the like to form baffles or compartments within the aneurysm 10. For example, FIG. 11 illustrates an expanded heat-set shape of a prosthesis component or layer 160 in which a plurality of radial, sinusoidal-like undulations 162 are formed.

In some embodiments, the undulations 162 may create barriers or compartments between the aneurysm and additional layers within the component 160. In one example, the undulations 162 create a plurality of sub-volumes that occupy between about 20% and 90% of the total aneurysm volume 170 (i.e, the volume of the entire aneurysmal artery segment less the volume of an extension of the natural artery(s) 174 through the aneurysm. In some embodiments, one or more components may substantially reconstruct the artery lumen thus forming one or more layers generally along the artificial lumen through the aneurysm.

FIG. 13 illustrates another prosthesis component 180 having a distal end 184 sized for a normal section of the aorta and a larger, dilated portion 182 so as to substantially conform and line the wall of the aneurysm 10. The dilated portion 182 may have an over-sized diameter so that its relaxed state is larger than the largest diameter of the aneurysm 10. Lining of the aneurysm wall may provide a fibrotic response and result in a strengthening of the aneurysm wall thus inhibiting rupture. Thus, while not reconstructing the natural artery lumen, it may still prevent rupture and protect the patient. Therefore, this component may act as a stand-alone, single-layer prosthesis or can be combined with any of the other components discussed in this specification.

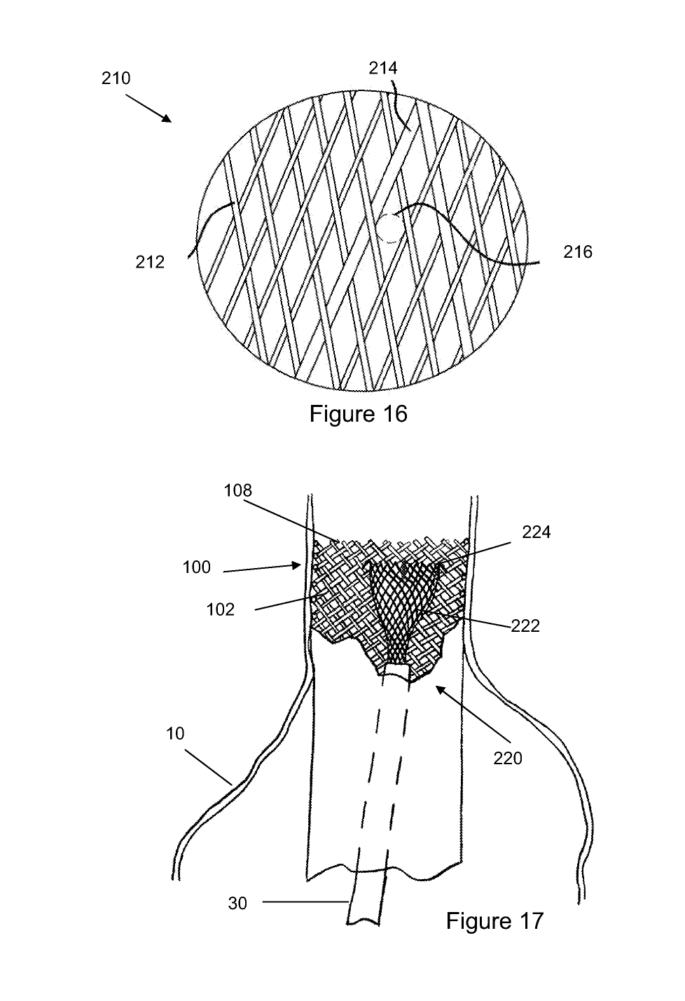

In some embodiments, one or more engagement members may be incorporated into one of more of the device layer(s). The engagement members may comprise hooks, barbs, tines, or other like elements designed to engage either the vessel wall and/or a previously placed stent or graft or device layer. In one example, engagement members 224 protrude outwardly from a distal end of component 222. As the component 222 is deployed, the engagement members 224 penetrate into or hook onto a previously placed component layer 100 as shown in FIG. 17. In another example, engagement members 224 protrude towards the vessel to penetrate into the vessel wall or provide an anchoring spring force. Thus, the engagement members 224 may promote fixation and stabilization of a first layer and/or a second layer and inhibit migration or dislodgement within a vessel.

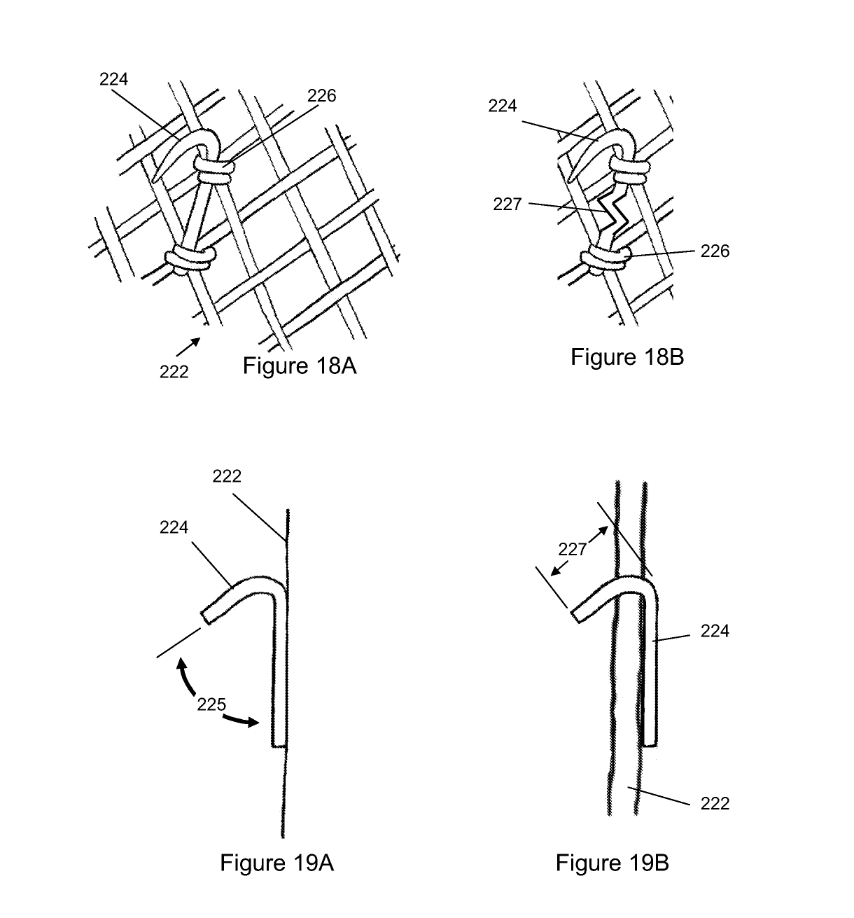

The engagement member(s) may be formed of a wire, filament or machined or cut piece to comprise a hook, prong or protruding tine that may be attached to a braided layer at one or more points. In some embodiments, the engagement member 224 is a hook that is attached at two or more points 226 as shown in FIG. 18A. Since the filaments of mesh component 222 move relative to each other during their deployment and expansion, the attachment points are configured so as to prevent any interference with this movement and mesh expansion. For example, the attachment points 226 can each be fixed to parallel filaments and allowed to slide along each filament during the component's expansion. In another example, the attachment points 226 can be slidingly-fixed on the same filament so as to overlap a crossing filament. In another example shown in FIG. 18B, a middle portion of the engagement member 224 between each attachment point 226 includes a region 227 that can increase or decrease in length (e.g., a plurality of alternating folds or bends that can increase or decrease in angle), thereby allowing each end of the engagement member 224 to move relative to each other.

The engagement member 224 may have a tapered or pointed end to facilitate tissue penetration or have a blunt end so as to minimize tissue penetration. The engagement members 224 may be adhered to a layer by various means known in the art including welding, laser welding, brazing, soldering, adhesives and the like. In some embodiments, one or more attachment members may be used to attach the engagement member(s) to a layer or filaments that comprise a layer. The engagement members may be polymeric or metallic filaments that are wrapped, tied, welded or bonded to the engagement member(s) at one or more points.

For some embodiments, the engagement member(s) 224 may form an angulated prong portion with respect to the layer 222, stent or graft surface to which it is attached as shown in FIG. 19jA. In some embodiments, the angle 225 may be an acute angle and in some embodiments, the angle may be between about 30° and 80°. The prong portion of the engagement member 224 may have a length 227 as shown in FIG. 19B that may be between about 2 mm and 6 mm. As can be appreciated in FIGS. 19A and B, the engagement members 114 can be attached to an inner or outer surface of a component.

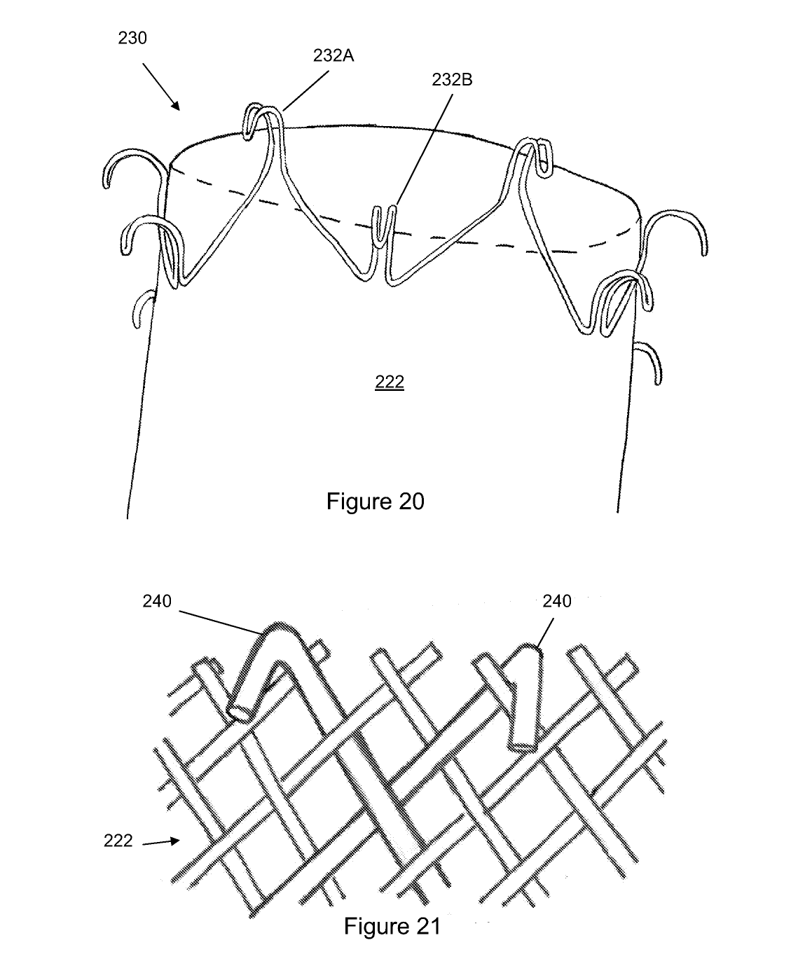

In some embodiments, a structure may be formed to provide an array of engagement members that form a ring about a portion of a layer or graft. For example, FIG. 20 illustrates an engagement ring 130 forming a zig-zag-like or wave pattern to facilitate collapse of the structure in concert with the collapse of the braid for delivery and retraction. In this respect, the ring forms a plurality of engagement members 232A at the “peaks” of the wave shape and a plurality of engagement members 232B at the “troughs” of the wave shape. However, it should be understood that the engagement members can be located at any position along the ring 230.

In another example seen in FIG. 21, engagement members 140 can be formed from the ends of one or more filaments of a mesh 222. These engagement members 140 can be formed filaments that are generally larger than or equal to those not forming engagement members 240.

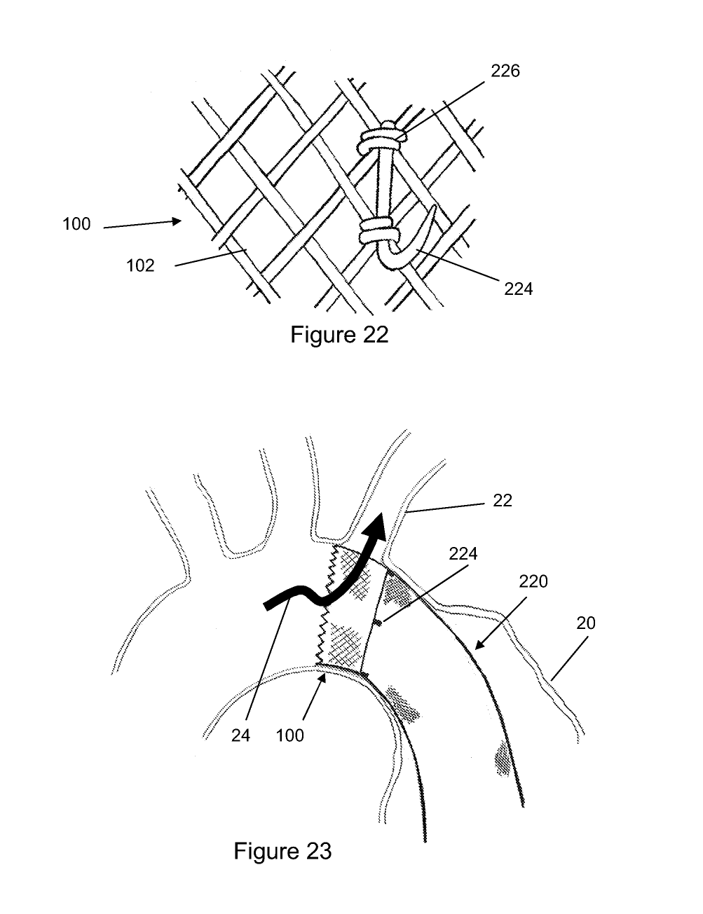

In another example shown in FIG. 22, an engagement member 224 can be located on the interior of a prosthesis layer 100 and pointed distally or upstream. In this regard, a first prosthesis layer 100 may be deployed with the engagement members 224 and a second prosthesis layer or component can deployed over and engaged with the engagement members 224. In another example, a first prosthesis layer may include a first set of internally-facing engagement members 224 and a second prosthesis layer may include a second set of externally-facing engagement members 224 so that each layer may engage each other. In yet another example, a prosthesis layer may include both internal and external attachment members 224 if located between two other prosthesis layers or between a vessel wall and a prosthesis layer. The engagement member may have undulations or other means to adjust in length to accommodate the mesh filaments on collapse.

In some embodiments, the prosthesis device may comprise multiple zones or regions of different density or porosity. For example, the device may have a first zone that has low density (or high porosity) to allow a sufficient amount of blood to flow through the wall so that a branch vessel remains patent. Further, the device may have one or more high density (or low porosity) regions or zones that allow minimal flow through the wall to enhance the occlusion and/or healing of a vascular defect such as an aneurysm or dissection. The different zones may be accomplished by the overlapping of different braid layers or the joining of different braids in and end-to-end fashion. The different zones may also be accomplished by changing the braid angle, number of wires, wire size, heat setting filaments to a favorable position, and other techniques described in this specification and known in the art.

In one example, a prosthesis device is used for the endovascular treatment of thoracic aneurysms also known as thoracic endovascular aneurysm repair or TEVAR as shown in FIG. 23. A first prosthesis layer 100 having a low density region may be deployed to cover the left subclavian artery 22 thereby allowing blood flow 24 through the exposed area of the layer 100. This may be particularly useful when the thoracic aneurysm 20 is within close proximity (e.g. about 15 mm) of the LSA junction resulting in a small landing area for a traditional stent graft. Small stent graft landing area can mean higher risk of improper positioning and device migration. A second, higher density layer 220 spans the aneurysm 20 downstream of the exposed portion of the low density layer 100.

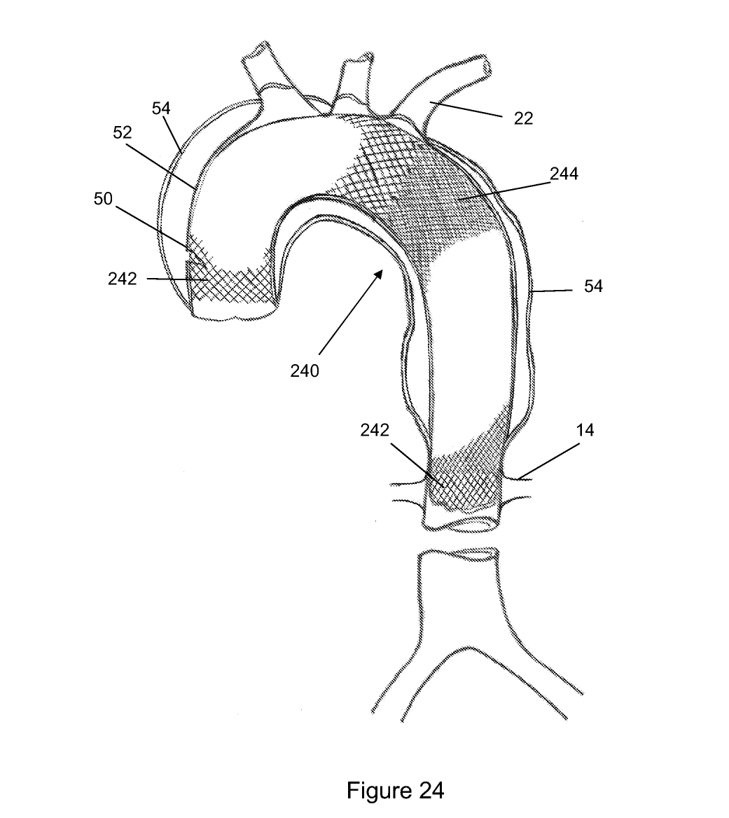

In another example shown in FIG. 24, a single prosthesis layer 240 is shown having two low density regions 242 at the proximal and distal ends of the device 240 and a high density region 244 along a middle region of the device 240. In this regard, the low density regions 242 can be deployed over subclavian arteries 22 and the renal arteries 14 to allow blood flow through the region 242 and into the arteries 14 and 22, while the high density region 244 is located over the thoracic dissection 50 and/or other areas of the thoracic aorta wall 52 that require reinforcing due to blood flow between the wall 52 and outer aorta layers.

Optionally, the lower density layer 100 may extend to a downstream position to cover, for example, the renal arteries (not shown). In some embodiments, the low density region may have an average maximum pore size of between about 200 microns and 2000 microns, in other embodiments between about 300 microns and 1,500 microns, and in yet other embodiments between about 250 and 500 microns. The higher density region may have an average maximum pore size of between about 100 microns and 300 microns and in some embodiments between about 150 and 250 microns.

Different layers of mesh layer may have different filament counts. In some embodiments, a layer may comprise a braided filament count greater than 300 filaments or ends. In one embodiment, the braided filament count for high density region is between about 360 to about 780 filaments, or in further embodiments between about 180 to about 640 filaments. In one embodiment, the braided filament count for a low density region is between about 140 and about 280 filaments, or in other embodiments between about 120 and about 200 filaments. In some embodiments, the device may include polymer filaments or fabric within the layers or between layers of braids.

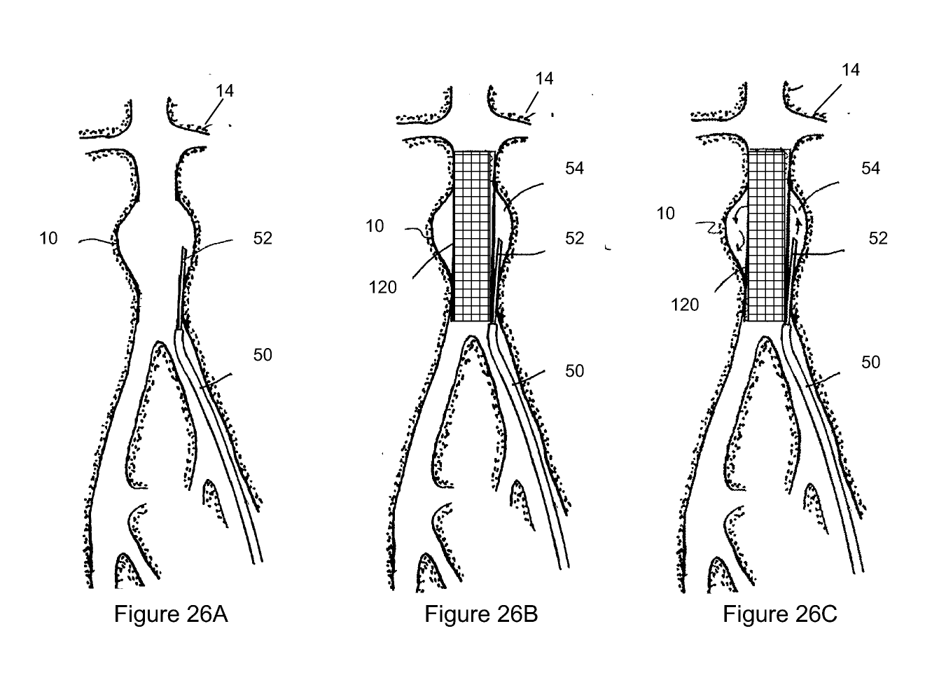

As seen in FIGS. 26A through 26C, the treatment procedure may include the injection or delivery of embolic material 54 or other space filling devices that may serve to reduce leakage through or around the device and may help stabilize the prosthesis. In FIG. 26A, a catheter 50 is percutaneously inserted into a femoral artery and advanced to a position where the distal end of the catheter 50 is located within the aorta slightly inferior to the aneurysm 10. A blunt tipped cannula 52 is then advanced out of the end of the catheter 50, into the aneurysmal portion of the aorta.

As shown in FIG. 26B, a straight prosthesis device 20 is then introduced, radially expanded and implanted. When so implanted, the prosthesis 120 bridges or extends through the aneurysm 10 and the ends of the prosthesis 120 are in substantial coaptation with the healthy aortic wall above and below the aneurysm 10. The blunt tipped cannula 52 is captured between the inferior end of the prosthesis 120 and the aorta wall, as shown. Preferably, the blunt tipped cannula 52 is formed of metal hypotubing or plastic tubing that is sufficiently strong and crush resistant to avoid substantial collapsing or closing of its lumen when it is compressed between the adjacent prosthesis 120 and the aorta wall, as shown in FIG. 26B. In some embodiments, the embolic or space filling material may be an unreacted monomer or polymer that is reacted or polymerized in vivo.

Thereafter, as shown in FIG. 26C, embolic or space filling material 54 (such as expansile polymeric material) is then injected through the catheter 50, through the lumen of the cannula 52, and into the aneurysm. After being introduced into the aneurysm, the embolic or space filling material 54 substantially fills the aneurysm sac 10. The catheter 50 and cannula 52 are then removed, leaving the prosthesis 120 and the embolic or space filling material 54 in place.

Additional details regarding the apparatus and methods of injection of materials between a vascular prosthesis and an aneurysm wall are described by Rosenbluth et al. in U.S. Patent Applications 2005/0004660 and 2003/0014075 both of which are herein incorporated in their entirety. As described therein, expansible materials such as foams or hydrogels may be used to fill spaces created by the mesh layers.

In some embodiments, the porous, multi-layer device comprises a self-expanding braided mesh containing at least one of the following materials: nickel-titanium alloys (e.g. Nitinol), stainless steel, alloys of cobalt-chrome, Elgiloy, 35N LT, Dacron, polyester, Teflon, PTFE, ePTFE, TFE, polypropylene, nylon, TFE, PET, TPE, PGA, PGLA, or PLA. Polymer materials described herein may provide a mild inflammatory response when implanted and may therefore enhance the embolization and healing of an aneurysm.

Optionally, the porous, multi-layer device may be constructed to provide the elution or delivery of one or more beneficial drug(s) and/or other bioactive substances into the blood or the surrounding tissue. Optionally, the device may be coated with various polymers to enhance its performance, fixation and/or biocompatibility. Optionally, the device may incorporate cells and/or other biologic material to promote sealing, reduction of leak or healing. In any of the above embodiments, the device may include a drug or bioactive agent to enhance the performance and/or healing of the device, including: an antiplatelet agent, including but not limited to aspirin, glycoprotein IIb/IIIa receptor inhibitors (including, abciximab, eptifibatide, tirofiban, lamifiban, fradafiban, cromafiban, toxifiban, XV454,lefradafiban, klerval, lotrafiban, orbofiban, and xemilofiban), dipyridamole, apo-dipyridamole, persantine, prostacyclin, ticlopidine, clopidogrel, cromafiban, cilostazol, dibigitran and nitric oxide. In any of the above embodiments, the device may include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, and thromboxane A2 receptor inhibitors.

In some embodiments, the inner layer(s) may be coated or have an enhanced surface to inhibit platelet and thrombus attachment while the external layer(s) may have a clot promoting agent or surface treatment. This combination of inhibition of luminal thrombus and promotion of external thrombosis may have a synergistic effect on treatment of an aneurysm or other vascular defect.

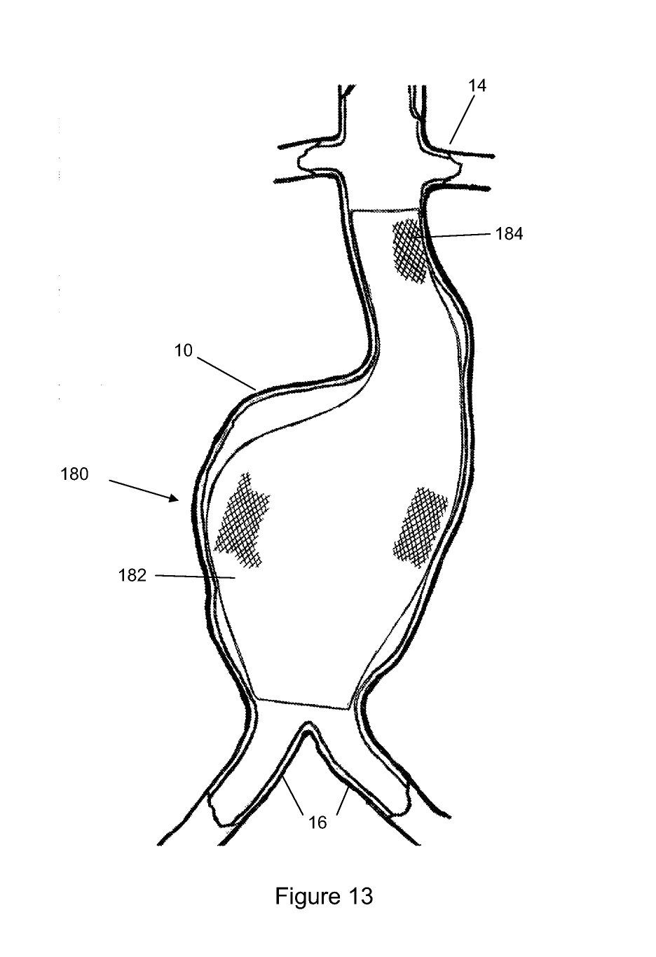

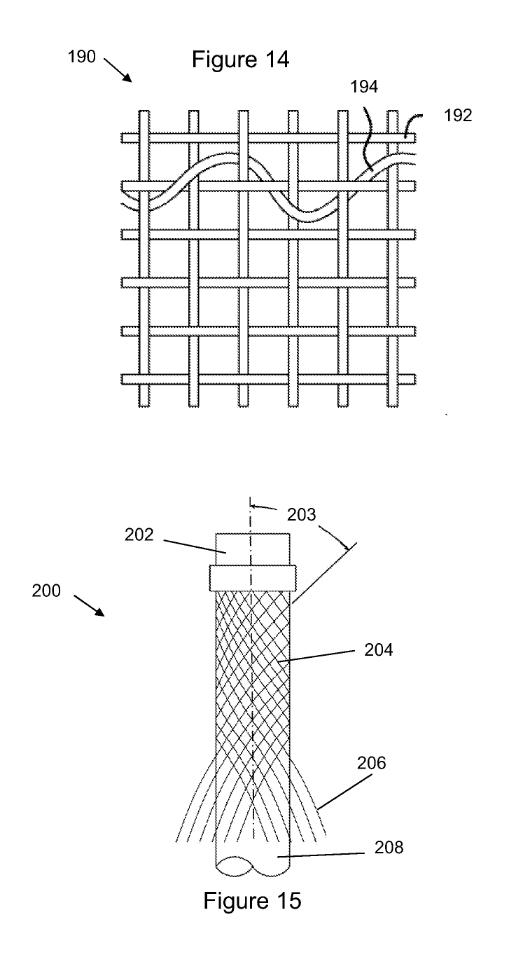

As seen in FIG. 14, the mesh 190 of any of the embodiments may include one or more filaments 194 interwoven with the mesh fibers 192 to provide for the delivery of drugs, bioactive agents or materials with a mild inflammatory response as disclosed herein. These interwoven filaments 194 may provide accelerated occlusion and/or enhanced healing of the mesh layers. The interwoven filaments 194 may be woven into the mesh structure 190 after heat treating to avoid damage to the interwoven filaments 192 by the heat treatment process.

The mesh may be fabricated from an integrated laser cut, mechanically cut or chemically etched structure, braided tubular wire mesh or combinations thereof. In one embodiment the distal portion of the mesh is held to the inner wall or the aorta by the radial and frictional forces of the expandable mesh. In other embodiments the distal portion of the mesh may incorporate structures to help provide additional fixation to the inner wall of the vessel. Structures may include at least one tine, barb, hook, pin or anchor (hereinafter called “barbs”). The length of the barbs may be from about 1 to 8 mm and preferably about 2 to 5 mm. Other structures at the distal portion may include the use of additional expandable wires, struts, supports, clips, springs, inflatable balloons, toroidal balloons, glues, adhesives or vacuum.

In some embodiments, the braided mesh of the previous embodiments may be formed over a mandrel 208 that also includes a braid-retaining collar 202 as is known in the art of tubular braid manufacturing and shown in FIG. 15. The braid angle 203 may be controlled by various means known in the art of filament braiding. The braids for the mesh 204 components may have a generally constant braid angle 203 over the length of a component or may be varied to provide different zones of pore size and radial stiffness.

Preferably, the ends of one or more components are configured in such a way as to prevent free filament ends from fraying. In one example, the filaments form a castellated braid that terminates one or more ends of the component with loops. This braiding technique is described in more detail in PCT Pub. No. WO2005/020822 to Moszner et al. and incorporated herein by reference. This braiding technique can also be seen in U.S. Pat. Nos. 5,824,040; 5,769,882; 6,110,198; and 7,481,822; each of which is incorporated by reference herein.

In one example, a first prosthesis layer having a low density has the same braid angle 203 as a second prosthesis layer having a high density and being located over the first prosthesis layer. The braid angle 203 of both layers may either be constant along their lengths or may vary together (i.e., both layers change braid angles 203 at the same locations and in the same amounts).

For braided portions, components, or elements, the braiding process can be carried out by automated machine fabrication or can also be performed by hand. For some embodiments, the braiding process can be carried out by the braiding apparatus and process described in U.S. Pat. No. 8,261,648, filed Oct. 17, 2011 and entitled “Braiding Mechanism and Methods of Use” by Marchand et al., which is herein incorporated by reference in its entirety.

In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments are loaded on the mandrel such that each filament extends radially toward the circumferential edge of the disc and each filament contacts the disc at a point of engagement on the circumferential edge, which is spaced apart at a discrete distance from adjacent points of engagement. The point at which each filament engages the circumferential edge of the disc is separated by a distance “d” from the points at which each immediately adjacent filament engages the circumferential edge of the disc.

The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating a second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement.

The tubular braided mesh 204 may then be further shaped using a heat setting process. As is known in the art of heat setting nitinol wires, a fixture or mold may be used to hold the braided tubular structure in its desired configuration and is then subjected to an appropriate heat treatment such that the resilient filaments of the braided tubular member assume or are otherwise shape-set to the outer contour of the mandrel or mold.

In some embodiments, the filamentary elements 206 of a mesh component 200 may be held by a fixture configured to hold the device or component in a desired shape and heated to about 475-525 degrees C. for about 5-15 minutes to shape-set the structure. In some embodiments, the braid may be a tubular braid of fine metal wires such as Nitinol, platinum, cobalt-chrome alloys, 35N LT, Elgiloy, stainless steel, tungsten or titanium. Alternate heat treatment cycles can be employed for different desirable mechanical properties in different materials. In some embodiments, the device can be formed at least in part from a cylindrical braid of elastic filaments. Thus, the braid may be radially constrained without plastic deformation and will self-expand on release of the radial constraint. Such a braid of elastic filaments is herein referred to as a “self-expanding braid.”

As discussed elsewhere in this specification, one or more components can be formed of a high density, substantially metal mesh that effectively seals a vascular defect. Many prior art devices require both a metal stent portion for structure and a non-stretchable fabric or polymer graft layer to seal a defect. Since these prior art fabrics or polymers are unable to stretch, they can prevent their stents, in the case of mesh, from axially elongating to its fullest extent. In contrast, the mesh embodiments of the present invention are not axially limited by fabric or other similar layers for forming a sealing layer, since the high density, low pore size mesh is capable of creating a sealing barrier by itself. Hence, the embodiments of the present invention are not only thinner during delivery in a catheter since they lack a second sealing layer, they can also axially elongate and therefore compress to a smaller radial diameter than many prior art devices.

For example, the thickness of the braid filaments may be less that about 0.3 mm. In some embodiments, the braid may be fabricated from wires with diameters ranging from about 0.02 mm to about 0.2 mm. In some embodiments, a device or component may have a high braid angle zone where the braid angle is greater than about 60 degrees.

In some embodiments, a device or component may have at least one zone or section where the radial stiffness is substantially higher than the remaining portion or section(s) of the device or component and the braid angle in the higher radial stiffness zone or section is greater than the average braid angle of the remaining portion or section(s) by at least about 10 degrees. In some embodiments with a higher stiffness zone or section, the higher stiffness zone or section may be less than about 25% of the overall length of the entire device or component.

In any of the embodiments described herein, one or more portions of a braided mesh 210 may form either zone or an entire component having small average effective pores 216 as shown in FIG. 16. In other words, these small pores form a mesh with a density high enough so as to ultimately prevent substantial passage of blood through and thereby seal a vascular defect. In some embodiments, the device or component may have at least one portion or zone with an average effective pore size 216 of less than about 300 microns (or 0.30 mm). In some embodiments, a component may have at least one portion or zone with an average effective pore size 216 between about 50 and 250 microns. In another embodiment, such a high density mesh has a density greater than about 70%. In another embodiment, such a high density mesh has a density greater than about 200 pics per inch. In this respect, these pore sizes or densities allow the mesh to function and thereby seal similar to fabrics and/or other polymer layers.

For some embodiments, three factors may be important for a woven or braided wire device for treatment of a patient's vasculature that can achieve a desired clinical outcome in the endovascular treatment of aneurysms. The inventors have found that for effective use in some applications, it may be desirable for the implant device to have sufficient radial stiffness for stability, limited pore size for rapid promotion of hemostasis leading to occlusion and a collapsed profile which is small enough to allow insertion through an inner lumen of a vascular catheter.

A device with a radial stiffness below a certain threshold may be unstable and may be at higher risk of movement or embolization in some cases. Larger pores between filament intersections in a braided or woven structure may not generate thrombus and occlude a vascular defect in an acute setting and thus may not give a treating physician or health professional such clinical feedback that the flow disruption will lead to a complete and lasting occlusion of the vascular defect being treated. Delivery of a device for treatment of a patient's vasculature through a standard vascular catheter may be highly desirable to allow access through the vasculature in the manner that a treating physician is accustomed.

The maximum pore size in a portion of a device that spans a vascular defect desirable for some useful embodiments of a woven wire device for treatment of a patient's vasculature may be expressed as a function of the total number of all filaments, filament diameter and the device diameter. The difference between filament sizes where two or more filament diameters or transverse dimensions are used, may be ignored in some cases for devices where the filament size(s) are very small compared to the device dimensions. For a two-filament device, the smallest filament diameter may be used for the calculation. Thus, the maximum pore size for such embodiments may be expressed as follows:

Pmax=(1.7/NT)(πD−(NTdw/2))
where Pmax is the average pore size,
D is the Device diameter (transverse dimension),
NT is the total number of all filaments, and
dw is the diameter of the filaments (smallest) in inches.

Using this expression, the maximum pore size, Pmax of the of some portions or components of the device may be less than about 0.016 inches or about 400 microns for some embodiments. In some embodiments the maximum pore size of some portions or components of the device may be less than about 0.012 inches or about 300 microns.

The collapsed profile of a two-filament (profile having two different filament diameters) woven filament device may be expressed as the function:

Pc=1.48((Nldl2+Nsds2))½
where Pc is the collapsed profile of the device,
Nl is the number of large filaments,
Ns is the number of small filaments,
dl is the diameter of the large filaments in inches, and
ds is the diameter of the small filaments in inches.

Using this expression, the collapsed profile Pc may be less than about 1.0 mm for some embodiments of particular clinical value. In some embodiments of particular clinical value, the device may be constructed so as to have both factors (Pmax and Pc) above within the ranges discussed above; Pmax less than about 300 microns and Pc less than about 1.0 mm, simultaneously. In some such embodiments, the device may be made to include about 70 filaments to about 300 filaments. In some cases, the filaments may have an outer transverse dimension or diameter of about 0.001 inches to about 0.012 inches.

In some embodiments, a combination of small filament 212 and large filament 214 sizes may be utilized (see FIG. 16) to make a device with a desired radial compliance and yet have a collapsed profile which is configured to fit through an inner lumen of commonly used vascular catheters. A device fabricated with even a small number of relatively large filaments can provide reduced radial compliance (or increased stiffness) compared to a device made with all small filaments. Even a relatively small number of larger filaments may provide a substantial increase in bending stiffness due to change in the moment of Inertia that results from an increase in diameter without increasing the total cross sectional area of the filaments. The moment of inertia (I) of a round wire or filament may be defined by the equation:

I=πd4/64
where d is the diameter of the wire or filament.

Since the moment of inertia is a function of filament diameter to the fourth power, a small change in the diameter greatly increases the moment of inertia. Thus, a small change in filament size can have substantial impact on the deflection at a given load and thus the compliance of the device. Thus, the stiffness can be increased by a significant amount without a large increase in the cross sectional area of a collapsed profile of the device. This may be particularly important as device embodiments are made larger to treat large aneurysms.

As such, some embodiments of devices for treatment of a patient's vasculature may be formed using a combination of filaments with a number of different diameters such as 2, 3, 4, 5 or more different diameters or transverse dimensions. In device embodiments where filaments with two different diameters are used, some larger filament embodiments may have a transverse dimension of about 0.004 inches to about 0.012 inches and some small filament embodiments may have a transverse dimension or diameter of about 0.001 inches and about 0.004 inches. The ratio of the number of large filaments to the number of small filaments may be between about 4 and 16 and may also be between about 6 and 10. In some embodiments, the difference in diameter or transverse dimension between the larger and smaller filaments may be less than about 0.008 inches, more specifically, less than about 0.006 inches, and even more specifically, less than about 0.003 inches.

This invention also comprises various methods of intravascular therapy. In particular, some embodiments describe methods for the endovascular treatment of abdominal aortic aneurysms (AAA) also known as endovascular aneurysm repair or EVAR.

For EVAR, in some method embodiments an introducer sheath is inserted over the primary access guidewire that has been introduced using vascular access techniques known in the art. A first sheath is advanced into one femoral artery, herein also referred to as the ipsilateral limb. If there is a concern about aortic rupture, a 12F sheath may be used in order to accommodate large diameter occlusion balloon. A 5F multipurpose catheter (e.g. Bern catheter) may be introduced to facilitate guidewire exchange to a stiff wire (such as the Meier wire or Amplatz). The stiff wire will straighten tortuosity of the access vessel and improve tracking capability of the introduced catheters and devices. An intravascular ultrasound (IVUS) catheter may be advanced over the stiff guidewire for inspection of the abdominal aorta. The use of IVUS allows the surgeon to interrogate the entire abdominal aorta and the iliac vessels and to map out (on the fluoroscopic screen) the renal and internal iliac arteries without the use of contrast/fluoroscopy. On the contralateral side, a 5F pigtail catheter may be used for angiography and may be introduced over the initial guidewire.

The curved end (e.g. pigtail) catheter may be used to perform an aortogram (i.e. an angiogram of the abdominal aorta) and the iliac arteries. After the angiogram is performed, the proximal neck may be evaluated. The length and the diameter of the proximal and distal neck may be measured using the preoperative CT scan and the intraoperative IVUS, as well as the angiogram. Based on the measurements taken, the sizes of the device components (lengths and diameters) may be chosen.

Introduction sheaths may be inserted into each femoral artery. Typically an introduction sheath between about 14 and 24F will be inserted in the ipsilateral limb and a 12-17F sheath on the other femoral artery, herein called the contralateral side. For this invention, a smaller introduction sheath may be feasible. In some embodiments, the introduction sheath or catheter for delivery of the device may be less than about 12F. In some embodiments, an introduction sheath or catheter for delivery of the device may be less than about 8F. As is known in the art of catheters, 3F is 1 mm and it generally refers to the lumen of the catheter.

If a device with a “pant leg” configuration is used (e.g., component 140 in FIG. 9A), the first device may be advanced into the proximal neck, positioned just inferior to the lowest renal artery 14 or just superior to the renal arteries 14, and oriented so that the contralateral limb gate can be easily accessed via the contralateral limb. The orientation of the contralateral gate may be performed with the aid of radiopaque markers under fluoroscopic guidance. The markers may be at the distal end of the trunk and/or on the stent graft bifurcation. A repeat angiogram may be performed to reconfirm the positioning of the device within the aorta. Subsequently, the first device is deployed and released.

In some method embodiments, the treatment procedure may include the injection or delivery of embolic material or other space filling materials or devices that may serve to reduce leakage through or around the device and may help stabilize the prosthesis. The material or devices may be injected or delivered between the layers and/or between the prosthesis and the arterial wall.

In some method embodiments, a multi-layer prosthesis is constructed to treat a vascular defect such as an aneurysm but the sequential delivery of a plurality of components through a catheter that is smaller than would be required if the device were constructed ex-vivo. Thus, the compressed radial profile of all of the components exceeds the luminal diameter of the delivery catheter.

In some method embodiments, the devices and/or components described herein may be used to create substantially closed structures or sub-zones of permeable mesh. Optionally, the closed structures or sub-zones may not be filled with a foreign body or material. Thus, they become filled with blood upon implantation and being substantially closed spaces, use the bodies own hemostasis and clotting mechanism to embolize the aneurysm volume. Accordingly, the devices and methods allow for a natural healing process to occur where the aneurysm may at least partially collapse or reduce in volume over time after treatment as the clotted blood organizes for form fibrous tissue. This can be advantageous compared to treatment where the aneurysm is substantially filled with devices, biomaterials or other foreign matter. In filled aneurysms, the biomaterials or foreign matter can impinge on tissues or organs in a similar manner to an untreated aneurysm and thus cause symptoms. Further, such devices, biomaterials or foreign matter can erode into other tissue structures or organs over time which can cause adverse consequences. In some embodiments, the closed spaces formed by the devices or components may comprise a volume between about 60% and 90% of the total aneurysm volume as defined herein and shown in FIG. 12.

In one method embodiment, a multi-layer prosthesis is constructed to treat an aneurysm proximate a bifurcation of an artery such that a first artery is upstream of the aneurysm and at least two branch vessels are downstream of the bifurcation. A first component is deployed wherein at least a portion of the aneurysm is lined with a mesh structure and a portion of the non-aneurysmal first artery is lined. A second component is deployed at least partly within the first component in a co-axial arrangement. At least one component is deployed extending from a previously placed component into a branch artery of the bifurcation. At least one component is deployed having at least a portion or zone with an average pore of less than about 250 microns and in some embodiments, the portion or zone has an average pore of less than about 150 microns.

In some method embodiments, a multi-layered vascular prosthesis is constructed using a plurality of mesh components such that at least one layer has at least a portion or zone that has an average pore of less than about 300 microns (or 0.3 mm). In some embodiments, at least one layer has at least a portion or zone that has an average pore between about 200 and 1,500 microns.

In any of the above embodiments, the device may include an antiplatelet agent, including but not limited to aspirin, glycoprotein IIb/IIIa receptor inhibitors (including, abciximab, eptifibatide, tirofiban, lamifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide. In any of the above embodiments, the device may include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, and thromboxane A2 receptor inhibitors.

A medical device is described comprising: a plurality of expandable mesh components that may be combined to form a multi-layer vascular prosthesis for the treatment of an aneurysm wherein more than about 60% of the aneurysm volume is encompassed by one or more closed structures comprising porous mesh. In some embodiments, all of the porous mesh components are formed of braided wire. In some embodiments, the expandable mesh components encompass between about 70% and 95% of the total aneurysm volume as defined herein and shown in FIG. 12.

A vascular prosthesis is described comprising at least one expandable mesh device for the treatment of an aneurysm wherein the aneurysm is protected from later rupture without the use of polymeric materials and can be delivered through a delivery catheter less than about 18F and in some embodiments between 10-18F. In some embodiments, the device is essentially all metallic materials which generally have lower thrombogenecity than polymeric materials. In addition, metallic filaments may be braided or otherwise fabricated into tubular mesh structures with the desired radial strength with a thinner and lower collapsed profile than polymeric materials thus allowing the use of a smaller introducer or delivery catheter.

Disclosed herein is a detailed description of various illustrated embodiments of the invention. This description is not to be taken in a limiting sense, but is made merely for the purpose of illustration of the general principles of the invention. Further features and advantages of the present invention will become apparent to those of skill in the art in view of the description of embodiments disclosed, when considered together with the attached drawings.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A vascular defect treatment prosthesis, comprising:
a first porous tubular component forming a first prosthesis layer expandable against a vessel;
a second porous tubular component forming a second prosthesis layer expandable against said first porous tubular component; said second porous tubular component being discrete from said first porous tubular component such that said second porous tubular component is consecutively deliverable after said first porous tubular component.

2. The prosthesis of claim 1, wherein said first tubular component further comprises an everted portion forming a torus shape around a non-everted cylindrical portion.

3. The prosthesis of claim 1, wherein said first porous tubular component comprises a first cylindrical portion and a second portion having a first fold directed radially outward from said first cylindrical portion so as to create an annular cavity around said first cylindrical portion.

4. The prosthesis of claim 3, wherein said second portion comprises a second fold directed radially inward such that an end of said first porous tubular component is located adjacent to said first fold.

5. The prosthesis of claim 1, wherein said second porous tubular component further comprises a cylindrical shape.

6. The prosthesis of claim 1, wherein said second porous tubular component further comprises a cylindrical shape having a first end portion being flared radially outward.

7. The prosthesis of claim 1, wherein said second porous tubular component further comprises a cylindrical shape having a first end portion forming a “D” shape.

8. The prosthesis of claim 1, further comprising a third porous tubular component; said third porous tubular component being discrete from said first porous tubular component and said second tubular component such that said third porous tubular component is consecutively deliverable after said first porous tubular component and said second porous tubular component.

9. The prosthesis of claim 1, wherein said second tubular component has a first cylindrical portion that bifurcates into a second cylindrical portion and a third cylindrical portion.

10. The prosthesis of claim 1, wherein said first tubular component has a heat-set expanded shape forming a plurality of radial diameter changes.

11. The prosthesis of claim 1, wherein said first tubular component has a heat-set expanded shape forming a first portion having a cylindrical shape with a first diameter and forming a second portion having a bulbous shape with a second diameter that is larger than said first diameter.

12. The prosthesis of claim 1, wherein said second porous tubular component further comprises a plurality of attachment members fixed near a distal end of said second porous tubular component; said attachment members engageable with said first porous tubular component upon subsequent deployment onto said first porous tubular component within a patient.

13. The prosthesis of claim 12, wherein said plurality of attachment members include hooks.

14. The prosthesis of claim 1, wherein said first porous tubular component further comprises a plurality of attachment members fixed near a distal end of said first porous tubular component; said attachment members engageable with said second porous tubular component upon subsequent deployment of said second porous tubular component onto said first porous tubular component within a patient.

15. The prosthesis of claim 12, wherein said plurality of attachment members are interconnected to form a ring around said second porous tubular component.

16. The prosthesis of claim 12, wherein said plurality of attachment members are formed from filaments interwoven into a mesh structure of said second porous tubular component.

17. The prosthesis of claim 1, wherein said first porous tubular component comprises a first portion having a first pore size and a second portion having a second pore size, wherein said first pore size is larger than said second pore size.

18. A method of treating an aortic aneurysm, comprising:
deploying a first tubular component across said aortic aneurysm; and,
subsequently deploying and expanding a second tubular component at least partially against said first tubular component.

19. The method of claim 18, wherein said deploying a first tubular component further comprising folding said first tubular component to form an outer everted portion and an inner cylindrical portion.

20. The method of claim 18, wherein said deploying a first tubular component further comprising deploying said first tubular component across a renal artery.

21. The method of claim 18, wherein said subsequently deploying and expanding a second tubular component further comprises deploying a portion of said second tubular component within an iliac artery.

22. The method of claim 18, further comprising subsequently deploying and expanding a third tubular component at least partially against said first tubular component.

23. The method of claim 18, wherein said subsequently deploying and expanding a second tubular component further comprises deploying a portion of said second tubular component within two iliac arteries.

24. The method of claim 18, wherein said deploying a first tubular component across said aortic aneurysm further comprises expanding said first tubular component into at least partial contact with a wall of said aortic aneurysm.

25. The method of claim 18, wherein said subsequently deploying and expanding said second tubular component further comprises engaging an engagement member of said second tubular component into engagement with said first tubular component.

26. The method of claim 18, wherein said subsequently deploying and expanding said second tubular component further comprises engaging an engagement member of said first tubular component into engagement with said second tubular component.

27. The method of claim 18, wherein said deploying a first tubular component further comprising deploying said first tubular component across a renal artery; and wherein said subsequently deploying and expanding said second tubular component further comprising deploying and expanding said second tubular component downstream of said renal artery.

28. The method of claim 18, further comprising injecting embolic material between said first tubular component and a wall of said aortic aneurysm.

29. A device for treating aortic aneurysms, comprising:
a first tubular component braided to expand to a first region having a first diameter and a second region having a second diameter that is larger than said first diameter; and,
a second tubular component being discrete from said first tubular component such that said second tubular component is consecutively deliverable after said first tubular component.

30. The device of claim 29, wherein said first tubular component is braided to form a plurality of pores having a first average pore size and said second tubular component is braided to form a plurality of powers having a second average pore size that is smaller than said first average pore size.

31. A vascular defect treatment prosthesis, comprising:
a first porous tubular component forming a first prosthesis layer expandable against a vessel; said first porous tubular component having an expanded state forming a first region having a first average pore size that is sized to substantially block passage of blood therethrough; and, a second region having a second average pore sized to allow passage of blood therethrough.

32. The prosthesis of claim 31, wherein said first average pore size is between about 100 microns and about 300 microns.

33. The prosthesis of claim 31, wherein said first average pore size is between about 150 microns and about 250 microns.

34. A vascular defect treatment prosthesis, comprising:
a first tubular mesh component formed substantially from a plurality of metallic filaments and forming a first prosthesis layer expandable against a vessel;
a second tubular mesh component formed from substantially from a plurality of metallic filaments and forming a second prosthesis layer expandable against said first porous tubular component; said second porous tubular component being discrete from said first porous tubular component such that said second porous tubular component is consecutively deliverable after said first porous tubular component.

35. The prosthesis of claim 1, wherein said first porous tubular component is substantially metallic and wherein said second porous tubular component is substantially polymeric.

36. The prosthesis of claim 1, wherein said first porous tubular component is composed of a plurality of metallic filaments and wherein said second porous tubular component is composed of a plurality of polymeric components.

37. The prosthesis of claim 1, wherein said second porous tubular component has a mesh structure forming an average pore size small enough to substantially prevent passage of blood.

38. The prosthesis of claim 1, wherein said second porous tubular component has a mesh structure forming an average pore size between about 100 and 300 microns.

39. The prosthesis of claim 1, wherein said second porous tubular component has a mesh structure forming an average pore size between about 150 and 250 microns.
